(19) United States
(12) Patent Application Publication
(10) Pub. No.: US 2007/0219576 A1
(43) Pub. Date: Sep. 20, 2007

(54) Reversibly and Radially Expandable Electroactive Polymer Element for Temporary Occlusion of a Vessel

(75) Inventor: Vincent J. Cangialosi, Beverly, MA (US)

Correspondence Address: MEDTRONIC VASCULAR, INC.;IP LEGAL DEPARTMENT, 3576 UNOCAL PLACE, SANTA ROSA, CA, 95403, US

(73) Assignee: Medtronic Vascular, Inc., Santa Rosa, CA (US)

(21) Appl. No.: 11/276,835

(22) Filed: Mar. 16, 2006

(51) Int. Cl. A61M 29/00

(52) U.S. Cl. 606/198

(57) ABSTRACT

An occlusion device is formed of an electroactive polymer element mounted on an elongated shaft. The electroactive polymer element includes an outer electrode, an inner electrode, and an electroactive polymer film disposed between the inner and outer electrodes. The electroactive polymer element may be formed in a hollow, generally cylindrical shape such that a longitudinal opening is formed inside of the inner electrode. The elongated shaft may be a guidewire or catheter. The inner and outer electrodes are coupled to an electrical power source such that an electrical voltage can be applied between the inner and outer electrodes. Upon application of the electrical voltage between the inner and outer electrodes, the electroactive polymer element expands to block a blood vessel of a patient.

### FIELD OF THE INVENTION

The invention relates generally to an intra-luminal occlusion device for blocking blood flow through a blood vessel of a patient.

### BACKGROUND OF THE INVENTION

Diseased blood vessels are a widespread medical condition. For example, a narrowing, or stenosis may form by local thickening of the vessel walls, or a lesion may form by an accumulation of atherosclerotic plaque on blood vessel walls. A thrombus (blood clot) may also form in a vessel, especially in a region of turbulent flow adjacent a narrowing. Blood vessel walls may also become thin and weak, possibly leading to the formation of an aneurysm. If a blood vessel becomes weakened or narrowed, clinical intervention may be required to prevent rupture or complete occlusion of the vessels. While many different surgical procedures are associated with the treatment of vascular diseases, the use of catheters is generally preferred due to the minimally invasive nature of interventional catheterization.

Many types of procedures involve the use of catheters to treat stenotic vessels or thromboses. One type of procedure is percutaneous transluminal coronary angioplasty, or PTCA, which involves the inflation of a balloon within a stenosis to expand a coronary blood vessel. Additionally, a stent may be implanted in conjunction with PTCA to support the dilated artery. Various other procedures are also common, such as a thrombectomy to remove a thrombus or a portion thereof, or an atherectomy to cut or abrade a stenosis within a diseased portion of the vessel.

Each of these intravascular procedures is associated with a common risk: that an embolic particle may be dislodged during the procedure and migrate through the circulatory system, possibly causing ischaemia, infarction or stroke. To prevent damage caused by such loosened debris, practitioners may attempt to capture the embolic particles using temporary distal protection devices such as occluder catheters or filter guidewires. Particles that are trapped or collected by such devices may be aspirated from the body lumen, chemically lysed in situ, or removed with the distal protection device at the end of the procedure.

Conventional occluder catheters may comprise a balloon catheter or mechanical occluder to block flow through the body lumen. Filter guidewires may comprise a filter to capture embolic material but permit blood flow through the body lumen. Filters may not prevent all of the debris from continuing downstream in the body lumen and may also require cumbersome and/or friction-prone arrangements for deployment of the filters. Balloons generally require a fluid to inflate the balloon to block the body lumen. These balloons risk leakage of the fluid. In addition, the fluid must be delivered to the balloon, generally through a separate lumen in the catheter. In order to provide prompt inflation and deflation of the balloon, the inflation lumen and the surrounding catheter shaft may be disadvantageously large. Further, additional complicated inflation-deflation hardware is required. Mechanical occluders also tend to require cumbersome supplemental apparatuses and may incorporate friction-prone push-pull mechanical arrangements. There is a need for a vessel occlusion device that is simple to construct and operate, and that can be expanded and contracted without requiring a large-diameter elongate shaft or an abundance of complicated accessory equipment.

### BRIEF SUMMARY OF THE INVENTION

The present invention is an occlusion device formed of an electroactive polymer element including an outer electrode, an inner electrode, and an electroactive polymer film disposed between the inner and outer electrodes. The electroactive polymer element may be formed in a hollow, generally cylindrical shape such that a longitudinal opening is formed inside of the inner electrode. The electroactive polymer element is mounted on a medical guidewire or catheter. The inner and outer electrodes are coupled to a power source such that an electrical voltage can be applied between the inner and outer electrodes. Upon application of the electrical voltage between the inner and outer electrodes, the electroactive polymer element expands to block the lumen of the blood vessel. When the electrical voltage is removed, the electroactive polymer element returns to its unexpanded state and blood flow can resume through the vessel.

The occlusion device of the present invention may be used in conjunction with a therapeutic interventional instrument such as a PTCA device, a stent, or a device for performing atherectomy, laser ablation, discectomy, or other similar procedures. When used with such a therapeutic interventional instrument, the occlusion device is located at a position relative to the treatment area, generally distally or downstream of the treatment area. The voltage is applied between the inner and outer electrodes, thereby causing the electroactive polymer element to expand into sealing apposition with the wall of the blood vessel to block blood flow there through. The therapeutic interventional procedure, such as implantation of a stent, is carried out while the electroactive polymer element is in its expanded state. The expanded electroactive polymer element blocks blood flow downstream of the treatment area. In particular, any embolic material that may come loose during the interventional procedure is prevented from proceeding downstream in the vessel. The area is aspirated to remove any embolic debris from the area. The voltage is then removed, thereby permitting the electroactive polymer element to return to its unexpanded state for removal from the vessel.

### BRIEF DESCRIPTION OF DRAWINGS

The foregoing and other features and advantages of the invention will be apparent from the following description of the invention as illustrated in the accompanying drawings. The accompanying drawings, which are incorporated herein and form a part of the specification, further serve to explain the principles of the invention and to enable a person skilled in the pertinent art to make and use the invention. The drawings are not to scale.

FIG. 1 is a perspective view of an electroactive polymer transducer of the prior art without electrical potential applied.

FIG. 2 is a perspective view of the transducer of FIG. 1 with an electrical potential applied.

FIG. 3 is a perspective view of an embodiment of an electroactive polymer element of the present invention without an electrical potential applied.

FIG. 4 is a perspective view of the electroactive polymer element of FIG. 3 with an electrical potential applied.

FIG. 5 is an elevational view, partially in section, depicting an embodiment of an occlusion device of the present invention disposed within a blood vessel of a patient, with the electroactive polymer element in an unexpanded state and an expandable interventional instrument (stent) in an unexpanded state.

FIG. 6 is an elevational view, partially in section, of the embodiment shown in FIG. 5, showing the electroactive polymer element in an expanded state.

FIG. 7 is an elevational view, partially in section, of the embodiment shown in FIGS. 5 and 6, showing the electroactive polymer element and the expandable interventional instrument both in their expanded states and embolic material released during the therapeutic interventional procedure.

FIG. 8 is an elevational view, partially in section, of the embodiment shown in FIGS. 5-7, including arrows indicating the direction of recovery of the embolic material, wherein the electroactive polymer element is in the expanded state.

FIG. 9 is an elevational view, partially in section, of the embodiment shown in FIGS. 5-8, wherein the electroactive polymer element is in the unexpanded state for removal from the vessel.

### DETAILED DESCRIPTION OF THE INVENTION

Specific embodiments of the present invention are now described with reference to the figures, where like reference numbers indicate identical or functionally similar elements. The terms “distal” and “proximal” are used in the following description with respect to a position or direction relative to the treating clinician. “Distal” or “distally” are a position distant from or in a direction away from the clinician. “Proximal” and “proximally” are a position near or in a direction toward the clinician.

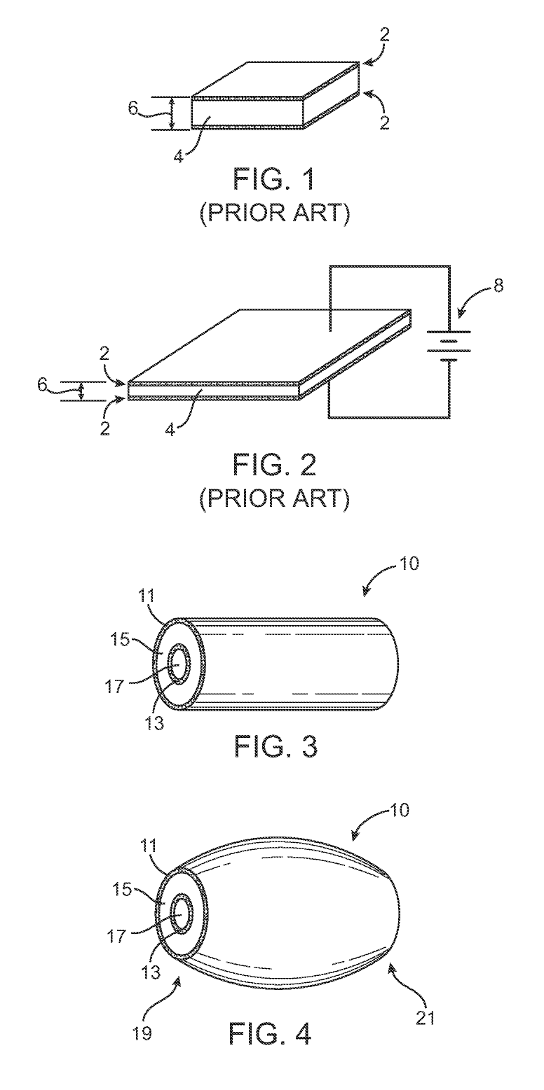

FIGS. 1 and 2 show a transducer of the prior art. In particular, the transducer is made up of electrodes 2 separated by an electroactive polymer (EAP) film 4. When the transducer of FIG. 1 is electrically charged by a power source 8, it deforms as shown in FIG. 2. The area increases and the thickness 6 decreases.

FIGS. 3 and 4 show an electroactive polymer element 10 of the present invention. Electroactive polymer element 10 is in the form of a hollow cylinder and includes an outer electrode 11, an inner electrode 13, and an electroactive polymer film 15 sandwiched between the electrodes. The hollow cylindrical shape of the electroactive polymer element 10 leaves a longitudinal opening 17 through the center of the electroactive polymer element 10. Electroactive polymer element 10 includes a proximal end 19 and a distal end 21.

Electroactive polymer film 15 may be any substantially insulating polymer or rubber or combination thereof that deforms in response to an electrostatic force or whose deformation results in or from a change in electric field. Exemplary materials include NuSil CF19-2186 made by NuSil Technology of Carpenteria, Calif., silicone polymers made by Dow Corning of Midland, Mich., acrylic elastomers such as VHB 4910 made by 3M Corp. of St. Paul, Minn., polyurethanes, thermoplastic elastomers, polymers comprising silicon and acrylic moieties, pressure-sensitive adhesives, fluoroelastomers, and the like. Thickness may range from 1 micrometer upwards. To increase the deformation capability, electroactive polymer film 15 can be pre-stretched, either directionally or isotropically. Films may be pre-stretched from 100 to 600%.

The term “electroactive polymers” refers to polymers that respond to electrical stimulation. Specific categories of electroactive polymers include electrostrictive polymers, electronic EAP and ionic EAP. Electrostrictive polymers are characterized by the non-linear reaction of an EAP relating to deflection. Electronic electroactive polymers change shape or dimensions due to migration of electrons in response electric field, usually dry. Ionic electroactive polymers change shape or dimensions due to migration of ions in response to an electric field, usually wet and including an electrolyte. Longitudinal electroactive polymers change in length in response to an electric field. Bending electroactive polymers respond to an electric field with bending (it may be the result of use of multiple layers as bimorph or an inherent property).

Outer electrode 11 and inner electrode 13 are compliant, flexible and expandable to maintain contact with electroactive polymer film 15 during deformation. Suitable materials may include graphite, carbon black, colloidal suspensions, thin metals including silver and gold, silver filled and carbon filled gels and polymers, and ionically or electrically conductive polymers. Structured electrodes may also be used, such as, metal traces and charge distribution layers, textured electrodes comprising out of plane dimensions. Suitable electrodes may also comprise conductive greases, such as carbon or silver greases and other high aspect ratio conductive materials such as carbon fibrils and carbon nanotubes and mixtures of ionically conductive materials.

Upon application of an electrical charge between outer and inner electrodes 11, 13, electroactive polymer element 10 deforms by expanding radially, as shown in FIG. 4, because proximal and distal ends 19, 21 are fixed to a shaft, as explained in detail below.

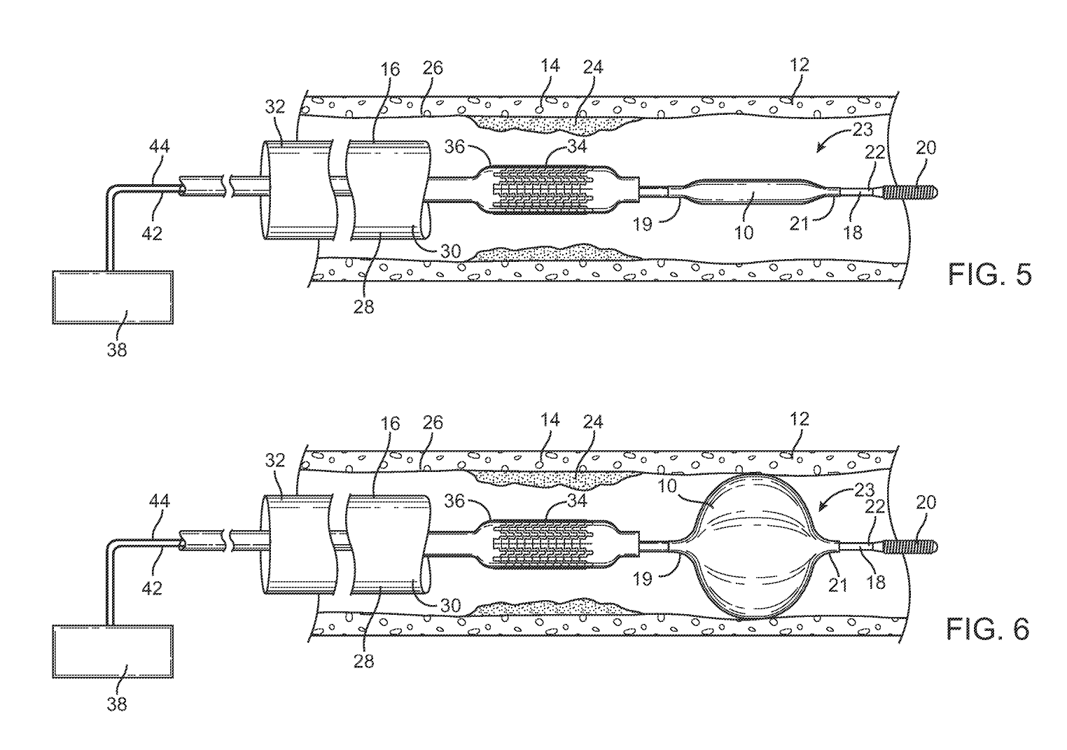

FIGS. 5-9 show an embodiment of an occlusion device 23 of the present invention provided for enabling an interventional procedure to be performed in a blood vessel 12 at a treatment area 14. Occlusion device 23 includes electroactive polymer element 10 mounted on an elongated shaft 18. Elongated shaft 18 may be a catheter, guidewire, or similar device. Elongated shaft 18 may include a coiled tip 20 at a distal end 22 thereof. As shown in FIGS. 5 and 6, a guide catheter 16 is provided that is adapted to guide the delivery of the elements for enabling the interventional procedure to be performed, and to guide the removal of the elements to be retrieved after performing the interventional procedure. Treatment area 14 may comprise atherosclerotic plaque 24 built up within/against the inside wall 26 of blood vessel 12, which locally decreases the inside diameter of blood vessel 12. As a result, blood flow may be diminished through this area. Guide catheter 16 may include an elongated shaft 28 having a distal end 30 and a proximal end 32 extending outside the patient's body.

The therapeutic interventional procedure may comprise positioning, expanding, and implanting an expandable interventional instrument, such as a stent 34, at treatment area 14, to dilate plaque 24 and wall 26 of blood vessel 12 to increase the inside diameter of treatment area 14 of blood vessel 12, and to help increase flow of blood to downstream vessels and organs. Stent 34 may be delivered to the interventional procedure site by a delivery system 36 extendable through guide catheter 16. The technique of implanting stent 34 may help increase the inside diameter of the occluded area, and towards this end, delivery system 36 may be adapted to enable stent 34 to be expanded and deployed at treatment area 14, for example, through balloon expansion. The continued presence of stent 34 may help prevent restenosis in treatment area 14, especially if stent 34 is a drug-eluting stent. Although occlusion device 23 of the present invention is shown with respect to a stenting procedure, occlusion device 23 can be used with any interventional procedure, such as balloon angioplasty (with or without stenting), or a device for performing atherectomy, discectomy, ablation or similar procedures.

As shown in FIG. 5, occlusion device 23 for occluding and blocking blood vessel 12 is positioned at a location relative to treatment area 14. Occlusion device 23 is shown in a so-called distal embolic protection location, wherein the device is deployed at a location distal to, or downstream of treatment area 14 in order prevent or block the flow of blood past the temporary occlusion and to enable the capture of embolic material 40 which may be entrained in the blood in blood vessel 12 during the therapeutic interventional procedure. Alternatively, occlusion device 23 may be deployed for “proximal embolic protection” (not shown) at a location proximal to, or upstream of a treatment area to cause hemostasis in the treatment area. Proximal embolic protection typically requires aspiration of the contaminated stagnant blood through a distal inlet opening in the occlusion device before the occlusion element is contracted to allow resumption of blood flow. In yet another alternative, occlusion devices may be deployed both distally and proximally of a treatment area to create an isolated chamber for interventional therapy within the vessel.

Occlusion device 23 comprises electroactive polymer element 10 mounted on elongated shaft 18. As discussed above, electroactive polymer element 10 of occlusion device 23 comprises an electroactive polymer film 15 sandwiched between inner and outer electrodes 13, 11. Electroactive polymer element 10 is delivered to its location relative to treatment area 14 in an unexpanded state, that is, without an electrical potential applied between inner and outer electrodes 13, 11, as shown in FIG. 5. Proximal end 19 and distal end 21 of electroactive polymer element 10 are mounted to elongated shaft 18. Electroactive polymer element 10 may be attached to elongated shaft 18 using UV curing acrylate blend adhesives, cyanoacrylate adhesives, two-part epoxies, or other biocompatible attachment means as would be known to those skilled in the art.

As shown in FIG. 6, electroactive polymer element 10 is expanded at a location relative to treatment area 14, generally downstream and distal of treatment area 14. Electroactive polymer element 10 is expanded by applying an electrical potential between inner and outer electrodes 13, 11 from a power source, shown schematically at 38. Inner and outer electrodes 13, 11 may be subjected to electrical charge through direct wiring 42, 44 coupled with suitable electronics for control of the stress and strain produced by electroactive polymer element 10. Wires 42, 44 may extend together or separately through one or more dedicated, and optionally insulative lumens in elongated shaft 18 for connection to respective inner electrode 13 and outer electrode 11 by solder, conductive adhesive or other means as would be apparent to those skilled in the art. Power source 38 may be an electrical grid or battery or any other device developing an electrical charge. Alternatively, outer and inner electrodes 11, 13 may be charged wirelessly by radiation energy sources of radio frequency (RF), microwave, ultrasound or other wavelengths.

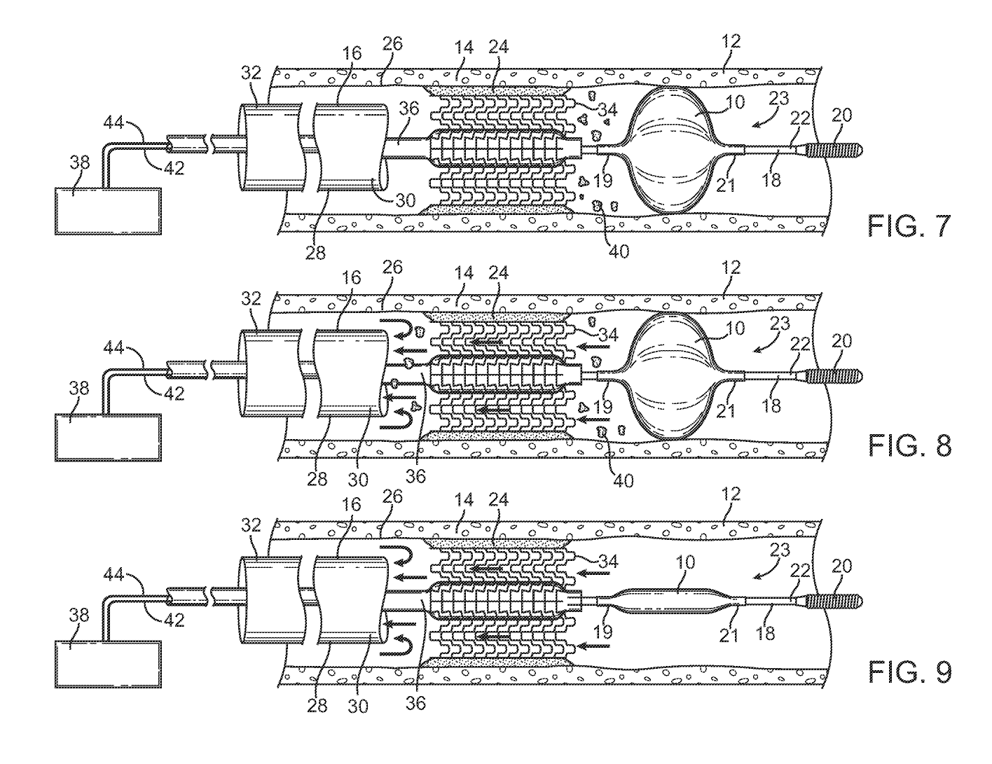

After occlusion device 23 is positioned relative to treatment area 14 and electroactive polymer element 10 is expanded, the therapeutic interventional procedure can be executed. The therapeutic interventional procedure shown in the drawings is simultaneous balloon angioplasty and stent delivery, which is known as direct stenting. Therefore, as shown in FIG. 7, stent 34 is expanded at treatment area 14. During crossing of treatment area 14 and expansion of stent 34, embolic material 40 may be released into the bloodstream. Embolic material 40 may be pieces dislodged from atherosclerotic plaque 24 at treatment area 14. Occlusion device 23 prevents embolic material 40 from proceeding downstream in vessel 12, where it may block smaller vessels downstream of treatment area 14. Accordingly, electroactive polymer element 10 preferably is non-porous to prevent and block blood flow through blood vessel 12. Optionally, the electroactive polymer element may be porous (not shown) to provide partial occlusion similar to a filter, thus trapping significantly large pieces of embolic material 40, while permitting minute debris and oxygenated blood to flow distally of treatment area 14.

During and after stent 34 is expanded, a valve (not shown) at a proximal end of guide catheter 16 is adapted to be opened to enable flushing and/or aspiration of embolic material through guide catheter 16, as shown by the arrows in FIGS. 8 and 9. Common devices and methods for flushing and aspiration are known to those skilled in the art.

After embolic material 40 has been aspirated out of the bloodstream, the voltage from power source 38 is removed from inner and outer electrodes 13, 11, thereby permitting electroactive polymer element 10 to return to its unexpanded state, as shown in FIG. 9. Occlusion device 23 can then be removed through guide catheter 16. Aspiration may continue through removal of occlusion device 23, as shown by the arrows in FIG. 9, or aspiration may be stopped before or after electroactive polymer element 10 returns to its unexpanded state.

The various components may be joined by suitable adhesives such as cyanoacrylate based adhesives. Heat shrinking, heat bonding, or ultrasonic welding may also be employed where appropriate. Plastic-to-plastic or plastic-to-metal joints can be affected by a suitable cyanoacrylate adhesive. Variations can be made in the composition of the materials to vary properties as needed.

While various embodiments of the present invention have been described above, it should be understood that they have been presented by way of illustration and example only, and not limitation. It will be apparent to persons skilled in the relevant art that various changes in form and detail can be made therein without departing from the spirit and scope of the invention. Thus, the breadth and scope of the present invention should not be limited by any of the above-described exemplary embodiments, but should be defined only in accordance with the appended claims and their equivalents. It will also be understood that each feature of each embodiment discussed herein, and of each reference cited herein, can be used in combination with the features of any other embodiment. All patents and publications discussed herein are incorporated by reference herein in their entirety.

What is claimed is:

1. An occlusion device comprising:
an elongated shaft adapted for insertion into a blood vessel of a patient;
an electroactive polymer element comprising an inner electrode, an outer electrode, and an electroactive polymer film sandwiched between the inner and outer electrodes, the electroactive polymer element forming a hollow, cylindrical tube in an unexpanded state, wherein a proximal end and a distal end of the electroactive polymer element are mounted to the shaft; and
a power source coupled to the inner and outer electrodes,
wherein the electroactive polymer element is adapted to expand from the unexpanded state for delivery within the blood vessel to an expanded state adapted to block blood flow through the blood vessel upon application of an electrical voltage between the inner and outer electrodes.

2. The device of claim 1, wherein the electroactive polymer film is selected from the group consisting of silicone polymers, acrylic elastomers, polyurethanes, thermoplastic elastomers, polymers comprising silicon and acrylic moieties, pressure-sensitive adhesives, and fluoroelastomers.

3. The device of claim 1, wherein the inner and outer electrodes are selected from the group consisting of graphite, carbon black, colloidal suspensions, thin metals, silver-filled gels and polymers, carbon filled gels and polymers, ionically or electrically conductive polymers, metal traces, and charge distribution layers.

4. The device of claim 1, wherein the power source is coupled to the inner and outer electrodes using wires.

5. The device of claim 4, wherein the elongated shaft is a catheter and the wires are disposed in one or more lumens within the catheter.

6. The device of claim 4, wherein the power source is an electrical grid or battery.

7. The device of claim 1, wherein the power source is wirelessly coupled to the inner and outer electrodes.

8. The device of claim 7, wherein the power source is a source of radiation selected from the group consisting of radio frequency, microwave, and ultrasound.

9. The device of claim 1, wherein the device is used in combination with a therapeutic interventional procedure selected from the group consisting of percutaneous transluminal coronary angioplasty, stent implantation, stent implantation in conjunction with percutaneous transluminal coronary angioplasty, atherectomy, laser ablation, and discectomy.

10. The device of claim 1, wherein the elongated shaft is a guidewire.

11. A method for temporarily occluding a blood vessel of a patient, the method comprising the steps of:
inserting an occlusion device into the blood vessel, wherein the occlusion device comprises an elongated shaft and an electroactive polymer element, wherein the electroactive polymer element includes an inner electrode, an outer electrode, and an electroactive polymer film sandwiched between the inner and outer electrodes, a proximal end and a distal end of the electroactive polymer element being mounted to the shaft;
advancing the occlusion device to a desired location in the blood vessel relative to a treatment area;
applying an electrical charge between the first and second electrodes such that the electroactive polymer element expands from an unexpanded state to an expanded state in which the electroactive polymer element blocks blood flow in the blood vessel;
removing the electrical charge from the first and second electrodes such that the electroactive polymer element returns to the unexpanded state; and
withdrawing the occlusion device from the blood vessel.

12. The method of claim 11, further comprising the step of conducting a therapeutic interventional procedure at the treatment area after the electroactive polymer element has been expanded to the expanded state.

13. The method of claim 12, wherein the therapeutic interventional procedure is selected from the group consisting of percutaneous transluminal coronary angioplasty, stent implantation, stent implantation in conjunction with percutaneous transluminal coronary angioplasty, atherectomy, laser ablation, and dissectomy.

14. The method of claim 12, wherein the desired location is distal to the treatment area.

15. The method of claim 1, further comprising the step of aspirating an area of the blood vessel proximal of the expanded electroactive polymer element.

16. The method of claim 11, wherein the electroactive polymer element is a hollow, cylindrical shape in the unexpanded state.

17. The method of claim 11, wherein the electroactive polymer film is selected from the group consisting of silicone polymers, acrylic elastomers, polyurethanes, thermoplastic elastomers, polymers comprising silicon and acrylic moieties, pressure-sensitive adhesives, and fluoroelastomers.

18. The method of claim 11, wherein the inner and outer electrodes are selected from the group consisting of graphite, carbon black, colloidal suspensions, thin metals, silver filled gels and polymers, carbon filled gels and polymers, ionically or electrically conductive polymers, metal traces, and charge distribution layers.

19. The method of claim 11, wherein the step of applying an electrical charge to the first and second electrodes comprises providing a power source outside the patient's body and coupling the power source to the inner and outer electrodes.

20. The method of claim 19, wherein the power source is coupled to the inner and outer electrodes using wires, wherein the elongated shaft is a catheter and the wires are disposed in at least one lumen within the catheter.

21. The method of claim 19, wherein the power source is coupled to the inner and outer electrodes wirelessly.
